(19) United States
(12) Patent Application Publication
(10) Pub. No.: US 2021/0059834 A1
(43) Pub. Date: Mar. 4, 2021

(54) MEDICAL IMPLANT HAVING AN ANCHORING SYSTEM AND METHOD OF USE THEREOF

(71) Applicant: Alliance Partners LLC, San Antonio, TX (US)

(72) Inventors: Natalie MIGUEL, San Antonio, TX (US); Bruce RICEMAN, Leander, TX (US); Brad FREDIN, Buda, TX (US); Anish VAGHELA, Boerne, TX (US)

(73) Assignee: Alliance Partners LLC, San Antonio, TX (US)

(21) Appl. No.: 17/003,850

(22) Filed: Aug. 26, 2020

Related U.S. Application Data
(60) Provisional application No. 62/891,888, filed on Aug. 26, 2019.

(51) Int. Cl. A61F 2/44; A61F 2/30; A61F 2/46

(52) U.S. Cl. CPC A61F 2/4455; A61F 2/447; A61F 2/30749; A61F 2/4611; A61F 2002/30841; A61F 2002/4633; A61F 2002/30224; A61F 2002/30784; A61F 2002/3093; A61F 2002/30405; A61F 2002/30476

(57) ABSTRACT

A medical implant having an anchoring system for anchoring and retaining the medical implant (such as a cage) in place and method of use thereof. The implantable intervertebral device can be used with any interchangeable combination of (a) anchors and (b) screws (i.e., the surgeon/practitioner can use the same medical implant and elect which combination of anchors and screws to use for anchoring purposes). The medical implant can be placed between the vertebrae and a tool be utilized to secure and anchor the medical implant in place.

### CROSS-REFERENCE TO RELATED PATENTS/APPLICATIONS

This application claims priority benefits to U.S. Patent Application Ser. No. 62/891,888, entitled “Medical Implant Having An Anchoring System And Method Of Use Thereof,” filed on Aug. 26, 2019. This patent application is commonly assigned to the Assignee of the present invention and is hereby incorporated herein by reference in its entirety for all purposes.

### FIELD OF INVENTION

A medical implant having an anchoring system for anchoring and retaining the medical implant (such as a cage) in place and method of use thereof.

### BACKGROUND OF INVENTION

The spine is the axis of the skeleton on which all of the body parts hang. In humans, the normal spine has seven cervical segments, twelve thoracic segments, five lumbar segments, five sacral segments (which fuse to form the sacrum) and three to five coccygeal segments (which fuse to form the coccyx. The lumbar spine attaches to the pelvis, and in turn is supported by the hip and leg bones. The bony vertebral bodies of the spine are separated by intervertebral discs, which act as joints but allow known degrees of flexion, extension, lateral bending, and axial rotation and translation.

Typical vertebra has a thick anterior bone mass called the vertebral body, with a neural (vertebral) arch that arises from the posterior surface of the vertebral body. The centers of adjacent vertebrae are supported by intervertebral discs. The disc and/or vertebral bodies may be displaced or damaged due to trauma, disease, degenerative defects, or aging over an extended period of time. One result of this displacement or damage to an intervertebral disc or vertebral body may be chronic back pain. In many cases, to alleviate back pain from degenerated or herniated discs, part or all of the disc is removed and may be replaced with an implant that promotes fusion of the remaining bony anatomy.

The success or failure of spinal fusion may depend upon several factors. For instance, the spacer or implant or cage used to fill the space left by the removed disc must be sufficiently strong to support the spine under a wide range of loading conditions. The spacer should also be configured so that it is likely to remain in place once it has been positioned in the spine by the surgeon. Additionally the material used for the spacer should be biocompatible material and should have a configuration that promotes bony ingrowth.

Other types of medical implants, such as corpectomy cages, have also been developed to help support the spine and maintain the normal spacing between opposing vertebrae. Typically, corpectomy cages are pre-manufactured at various heights requiring that a cavity between opposing vertebrae be prepared and distracted to a dimension corresponding to the most suitably sized corpectomy cage. The surgical procedure to prepare the implant site can be difficult and lengthy. Moreover, the procedure can increase risk of trauma to the tissues surrounding of the implant site.

Distractible corpectomy cages may be used as both a fusion device and/or a means for maintaining intervertebral spacing. Sometimes these implants include an actuation mechanism that allows the corpectomy cage to be expanded in situ to a size that corresponds to the cavity created when the damaged tissue is removed. These medical implants are referred to as “expandable medical implants” or “expandable cages.” The activation mechanism typically includes devices such as gears, threaded rods, and the like, in mechanical engagement so as to expand or contract the device to a necessary distance between the vertebrae. For medical implants that have cages that do not expand in situ, these are referred to as “non-expandable medical implants” or “non-expandable cages.”

Cervical cages are also used to stabilize the spine during the fusion process. An example of a cervical cage is the Alamo® Cervical Intervertebral Body Fusion device (Alliance Spine, San Antonio, Tex.), which is shown in FIG. 1. In FIG. 1, cage 100 is shown inserted in spinal column 101. Such cage 100 can be manufactured from PEEK Optima® LT1 (Invibio Biomaterial Solutions, West Conshohocken, Pa.) per ASTM F2026 and includes tantalum markers per ASTM F560 for radiographic visualization.

Furthermore, the cage can have the following features and benefits:

Length and width footprint can vary, such as 12 mm×14 mm, 14 mm×17 mm, and 16 mm×20 mm.

Heights can often vary in 1 mm increments from 5 mm to 12 mm (although these heights can be outside these ranges depending upon the patient).

The cage can have teeth on superior and inferior surfaces, which are designed to provide secure engagement and to prevent expulsion and migration.

The cage can be made of a biocompatible radiolucent polymer, which allows clear assessment of bony fusion. Alternatively, the cage can be made with a titanium alloy or a combination of a biocompatible radiolucent polymer and titanium alloy.

The cage can have a large graft area, which allows for optimal bone graft placement.

A top view of an exemplar intervertebral/interbody cage (cage 200) is shown in FIG. 2. For orientation purposes terms like “anterior,” “posterior,” “sagittal,” “superior,” and “inferior” are describing front, back, side, top, and bottom, respectively for the normal orientation of use of a medical implant, such as cage 200. As shown in FIG. 2, cage has anterior end 202, posterior end 201, lateral sides 203 and 204, superior face (which is the top view shown for cage 200 in FIG. 2), and inferior face (which is not shown in FIG. 2 as it is the underside of cage 200 as illustrate in this FIG. 2).

The “lateral” sides are positioned in a direction that is parallel to the plane of the sagittal view, i.e., the lateral sides are in a parasagittal plane. When lateral sides move outward (“lateral”) or inward (“medial”) relative to one another, these lateral sides are referred to as moving in a “lateral direction” or “medial direction,” respectively, in the normal orientation of use of an anterior medical implant of the present invention.

The “vertical” direction is the direction in the plane of the superior/inferior views, i.e., when the superior and inferior faces move upward/downward relative to one another, these superior and inferior faces are referred to as moving in a vertical direction. It should be noted that due to symmetry of many medical implants, the “superior” and “inferior” sides are interchangeable (in that the medical device can be flipped). Thus, the superior and inferior sides are relative to one another. The height of a medical implant is measured in the vertical direction. Moreover, when the medical implant has an expandable height, the expanding is done in the vertical direction.

As shown in FIG. 2, the cage does not expand either vertically or laterally, and therefore is a non-expandable cage.

U.S. Pat. No. 8,328,872 issued Dec. 11, 2012 to Duffield et al. (“Duffield”) discloses an intervertebral implant for implantation in a treated area of an intervertebral space between vertebral bodies of a spine, which includes a cage having screw holes extending from a side portion to the inferior (bottom) and superior surfaces (top) of the cage, in which the cage portion contains screws holes for receiving screws. There is also a screw back out prevention mechanism adapted on the plate portion and prevents the back out of screws from the screw holes. Illustration of the Duffield cage is shown in FIGS. 3A-3C. FIG. 3A is a perspective view of the cage 300. FIG. 3B is a sagittal view of cage 300. FIG. 3C is a perspective view of cage 300 with fasteners (screws 301) inserted. Other than changes in the reference numbers, FIGS. 3A-3C correspond to FIGS. 1, 3, and 7, respectively of Duffield. The screw holes of cage 300 are situated for receiving bone screws 301 that can be attached to the adjacent vertebral bodies at different angles to secure the cage in its position.

U.S. Pat. No. 9,044,337, issued Jun. 2, 2015 to Dinville et al. (“Dinville”) discloses an anchoring device and system for an intervertebral implant, intervertebral implant and implantation instrument. The anchoring device has a body include at least one curved plate elongated along a longitudinal axis, designed to be inserted through a passage crossing at least a part of implant, to penetrate into at least one vertebral endplate and attach implant onto this vertebral endplate by means of at least one stop retaining the implant, characterized in that the body includes at least one longitudinal rib (referred to as an “anchor”) on at least a part of at least one of its faces, the rib being designed to cooperate with a groove made in passage of implant. In some configurations, the anchoring device further withdrawal stops or latches, and/or means for withdrawing the anchor from an inserted position.

U.S. Pat. No. 8,545,562, issued Oct. 1, 2013 to Materna et al. (“Materna”) discloses a deployable member for use with an intervertebral cage. The implant has a body capable of being fit in an intervertebral space and a spin-plate rotatable with respect to the body. The spin-plate may a blade. The blade may possess grooves in its flat faces, or may be provided with an overhang. The path of the overhang or groove may be such that as the spin-plate advances in its rotation, a vertebra engaged with the blade is drawn toward the body of the implant. Pins may be placed in adjacent vertebrae such as to engage the spin-plate.

As the medical implant (cage) is subject to movement when the surgeon/practitioner is inserting and securing it, there is a need for an improved anchoring system that allows a surgeon/practitioner to more consistently and conveniently hold secure and anchor the medical implant in place.

### SUMMARY OF INVENTION

The present invention regards an improved cage that has an improved anchoring system. The present invention regards an implantable intervertebral device that can be used with any interchangeable combination of (a) anchors and (b) screws.

In general, in one aspect, the invention features a medical implant system. The medical implant system includes a plurality of fasteners including screws and/or anchors. The medical implant system further includes a receiving member body having a plurality of openings. Each of the openings in the plurality of openings is capable of interchangeably receiving a screw or an anchor from the plurality of fasteners. The medical implant system further includes anchor system instrumentation that is operable for rigidly attaching to the receiving member body resulting in the anchor system instrumentation being perpendicular to a midline of the receiving member body. The medical implant system includes a plurality of locking mechanisms connected to the receiving member body. Each opening in the plurality of openings has at least one locking mechanism located nearby. Each of locking mechanism in the plurality of locking mechanisms is capable of being moved with an instrument between an open position and a closed position relative to the opening nearby the locking mechanism. When the locking mechanism is in the open position, the locking mechanism does not cover any portion of the opening nearby the locking mechanism such that the fastener can be passed through the opening to secure the fastener into bone. When the locking mechanism is in the closed position, a portion of the locking mechanism covers a portion of the opening such that the fastener cannot pass through the opening, such that the fastener is prevented from becoming unsecured from the bone.

Implementations of the invention can include one or more of the following features:

The anchor system instrumentation can include one or more instruments selected from a group consisting of anchor inserters, anchor awls, anchor drivers, and combinations thereof.

The anchor system instrumentation can include an anchor inserter, an anchor awl, and an anchor driver.

The receiving member body can be interbody cage.

The anchors can have anchor blades.

The openings can be cylindrical.

The openings can have notches that orient the anchors during insertion into the receiving member body.

The openings and the anchors each can have a tab for visual conformation that the anchor has been seated in position within the opening.

The anchor can have anchor notches to prevent anchor movement after securing the anchor to the bone.

The anchor can have a surface that allows for a porous or textured surface for bone-in growth into the anchor.

The receiving member body can have a window so that a bone growth inducing substance can be utilized with the medical implant or so that fusion can be more readily viewed.

Each of the locking mechanisms can have an integrated anti-backout mechanism.

The anti-backout mechanism prevents the screw or blade of the anchor from migrating out of the interbody cage.

The anti-backout mechanism can be operable for interacting with the receiving member body to indicate the open position and the closed position.

In general, in another aspect, the invention features a method that includes the step of selecting a medical implant system having a plurality of fasteners, a receiving member body having a plurality of openings, and a plurality of locking mechanisms. The plurality of fasteners includes screws and anchors. The method further includes the step of selecting a first fastener from the plurality of fasteners. The first fastener is a screw or an anchor. The method further includes the step of, while a first locking mechanism is in an open position relative to a nearby first opening in the plurality of openings, inserting the first fastener through the nearby first opening such that the first fastener is received into the receiving member body. The method further includes the step of securing the first fastener into bone. The method further includes the step of repeating the above described steps for additional fasteners in the plurality of fasteners to secure the receiving member body. For each additional fastener, the additional fastener selected can independently be a screw or an anchor from the plurality of fasteners. The method further includes the step of moving the locking mechanisms from the open positions to the closed positions in which portion of the first locking mechanism covers a portion of the first opening such that the fasteners cannot pass through the openings, and such that the fasteners are prevented from becoming unsecured from the bone.

Implementations of the invention can include one or more of the following features:

The method can further include rigidly attaching anchor system instrumentation to the receiving member body resulting in the anchor system instrumentation being perpendicular to a midline of the receiving member body.

The anchor system instrumentation can include one or more instruments selected from a group consisting of anchor inserters, anchor awls, anchor drivers, and combinations thereof.

The anchor system instrumentation can include an anchor inserter, an anchor awl, and an anchor driver.

The receiving member body can be an interbody cage.

The anchors can have anchor blades.

The openings can be cylindrical.

The method can further include orienting the anchors during insertion into the receiving body utilizing one or more notches in the opening.

The method can further include visually confirming that the anchor has been seated in position within the opening utilizing tabs in the opening and the anchor.

The anchor can have anchor notches to prevent anchor movement after securing the anchor to the bone.

The anchor can have a surface that allows for a porous or textured surface for bone-in growth into the anchor.

The receiving member body can have a window. The method can further include utilizing the window to (i) provide a bone growth inducing substance, (ii) view fusion, or (iii) both.

Each of the locking mechanisms can have an integrated anti-backout mechanism.

The anti-backout mechanism can prevent the screw or anchor from migrating out of the interbody cage.

The anti-backout mechanism can interact with the receiving member body to indicate the open position and the closed position.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts a prior art cage inserted in a spinal column.

FIG. 2 depicts a prior art, non-expandable cage.

FIGS. 3A-3C depict different views of a prior art, non-expandable cage. FIG. 3A shows a perspective view. FIG. 3B shows a sagittal view. FIG. 3C shows a perspective view with fasteners.

FIGS. 4A-4F depict different views of an intervertebral device of the present invention in which anchors are inserted. FIGS. 4A-4D show the intervertebral device in the locked position, while FIGS. 4E-4F show the intervertebral device in the unlocked position.

FIGS. 5A-5F depict different views of the intervertebral device of the present invention shown in FIGS. 4A-4F in which screws are inserted. FIGS. 5A-5D show the intervertebral device in the locked position, while FIGS. 5E-5F show the intervertebral device in the unlocked position.

FIG. 6 depicts a view of the intervertebral device of the present invention shown in FIGS. 4A-4F and 5A-5F in which a combination of anchors and screws are inserted.

FIGS. 7A-7E depict different views of the intervertebral device of the present invention shown in FIGS. 4A-4F, 5A-5F, and 6 without any anchors or screws.

FIGS. 8A-8B depict the locking mechanism of the intervertebral device of the present invention.

FIG. 9A-9D depict the anchors that can be used in the intervertebral device of the present invention shown in FIGS. 4A-4F.

FIG. 10 depicts a view of another intervertebral device of the present invention in without any anchors or screws inserted.

FIG. 11A-11D depict the anchors that can be used in the intervertebral device of the present invention shown in FIG. 10.

FIG. 12 depicts an anchor inserter that is coupled to the intervertebral device of FIG. 10.

FIG. 13A depicts a magnified view of the head portion of the anchor inserter of FIG. 12 (with an uncoupled intervertebral device).

FIG. 13B depicts a cross section of the head portion shown in FIG. 13B (with an uncoupled intervertebral device).

FIGS. 13C-13D depict other magnified views of the head portion of the anchor inserter of FIG. 12 (with a coupled intervertebral device).

FIG. 14A depicts an anchor awl with the anchor inserter of FIG. 12.

FIG. 14B depicts a magnified view of a portion of the anchor awl shown in FIG. 14A (with an anchor inserter and coupled intervertebral device).

FIGS. 15A-15C depict, respectively, sections through the (i) anchor awl, (ii) anchor, and (iii) anchor awl and anchor showing profiles.

FIG. 16 depicts another magnified view of the portion of the anchor awl shown in FIG. 14A.

FIG. 17 depicts a magnified view of the anchor tip shown in FIG. 16.

FIG. 18A depicts an anchor driver with the anchor inserter of FIG. 12.

FIG. 18B depicts a magnified view of a portion of the anchor driver shown in FIG. 18A (with an anchor inserter and coupled intervertebral device).

FIGS. 19A-19C are other views of the anchor driver shown in FIG. 18A (fully coupled with an anchor inserter and intervertebral device).

FIG. 20 is a flowchart of a surgical technique using the intervertebral device of the present invention.

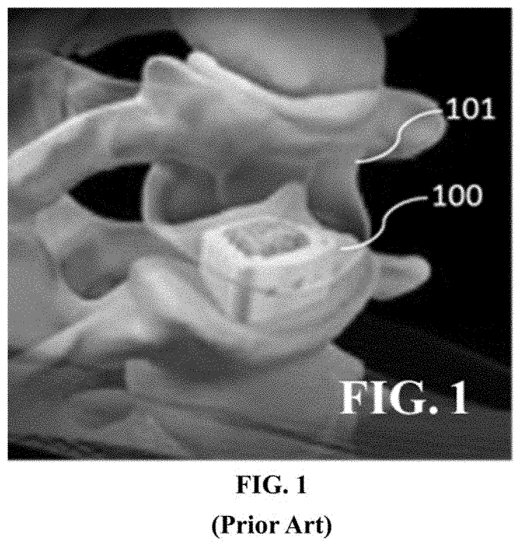

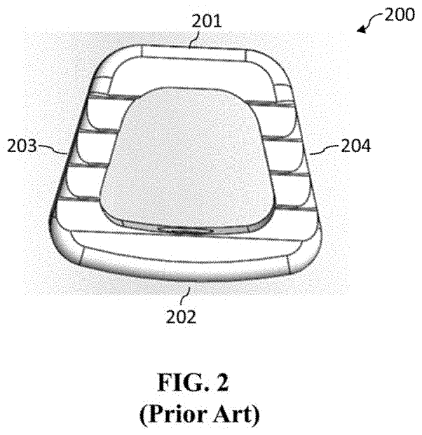

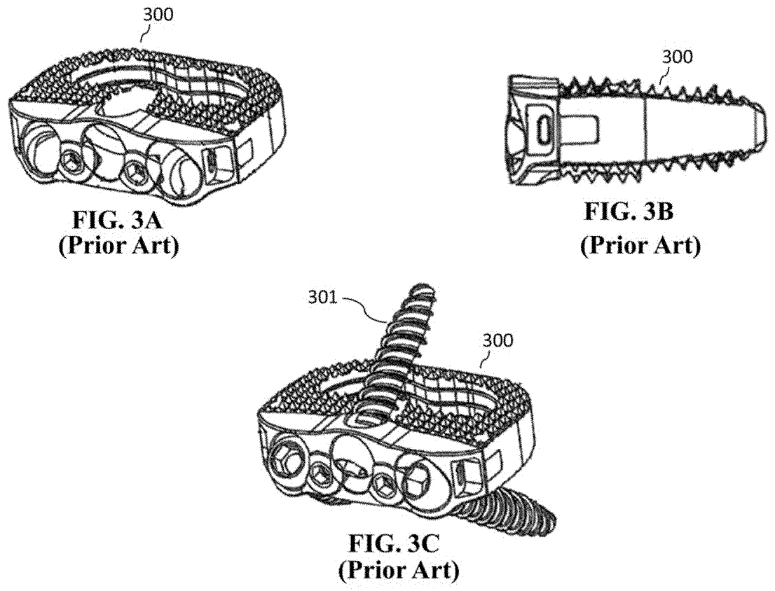

### DETAILED DESCRIPTION

The medical implant having an anchoring system for anchoring and retaining the medical implant (such as an interbody cage) in place and method of use thereof. The medical implant can be placed between the vertebrae and a tool can be utilized to secure and anchor the medical implant in place. The present invention regards an implantable intervertebral device that can be used with any interchangeable combination of (a) anchors and (b) screws (i.e., the surgeon/practitioner can use the same medical implant and elect which combination of anchors and screws to use for anchoring purposes).

The medical implant fixation instrument can also afford the surgeon/practitioner with graft windows so that the bone growth inducing substance can still utilized with the medical implant or so that fusion can be more readily viewed by the surgeon/practitioner.

While the figures refer to medical implant fixation system to be used for an anterior placement orientation, the medical implant fixation system can be used in orientation and with a variety of medical implant systems (such as with plates).

### Intervertebral Devices

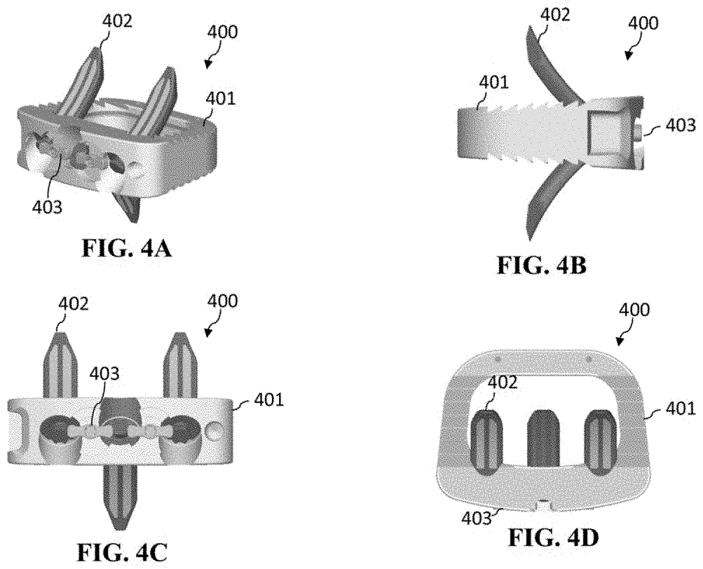

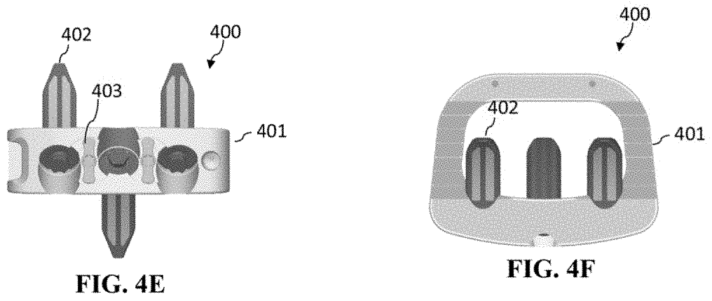

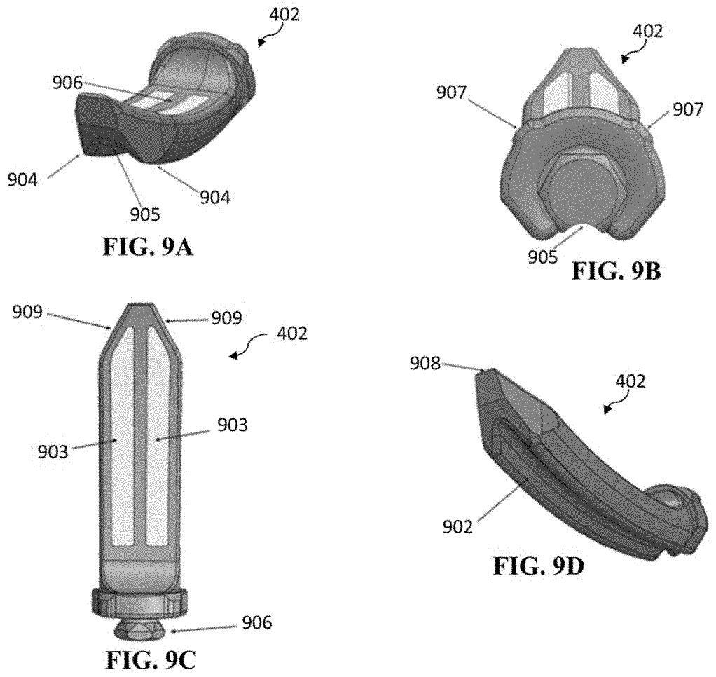

Referring to the figures, an embodiment of the present invention is depicted in FIGS. 4A-4F, 5A-5F, 6, and 7A-7E, which is an intervertebral device. FIGS. 4A-4F depict different views of an intervertebral device 400 in which anchors 402 are inserted. Embodiments of anchors 402 that can be used in the present invention are shown in FIGS. 9A-9D and are discussed below. Intervertebral device 400 has a body portion 401 (such as an interbody cage) in which the anchors 402 are received (and which can be used by a surgeon/practitioner to anchor the intervertebral device 400. Intervertebral device 400 also is shown to have a locking mechanism 403, which can be set in the open (unlocked) position or the closed (locked position). When the locking mechanism 403 is in the open position, the anchors 402 can be inserted into the intervertebral device and anchored by the surgeon. When the locking mechanism 403 is in the closed position, the anchors 402 are held in place and remain anchored. FIGS. 4A-4D show the intervertebral device 400 in the locked position, while FIGS. 4E-4F show the intervertebral device 400 in the unlocked position.

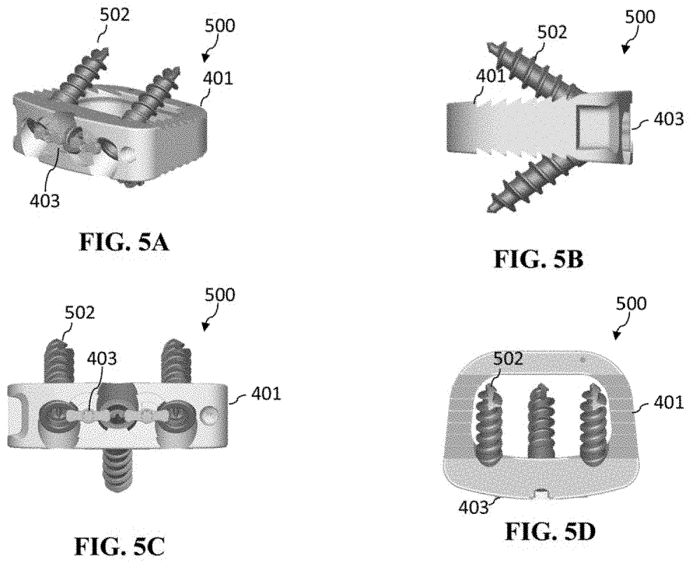

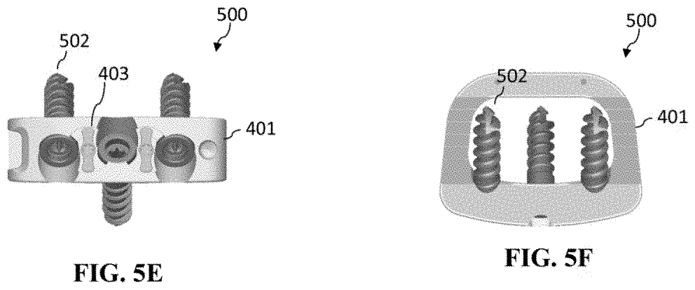

FIGS. 5A-5F depict different views of intervertebral device 500, which is the same as intervertebral device 400 except that, in place of anchors 402, it has screws 502 that are inserted. Such screws 502 can be standard screws as used in the art. FIGS. 5A-5D show the intervertebral device 500 in the locked position, while FIGS. 5E-5F show the intervertebral device 500 in the unlocked position. When the locking mechanism 403 is in the open position, the screws 502 can be inserted into the intervertebral device and anchored by the surgeon. When the locking mechanism 403 is in the closed position, the screws 502 are held in place and remain anchored.

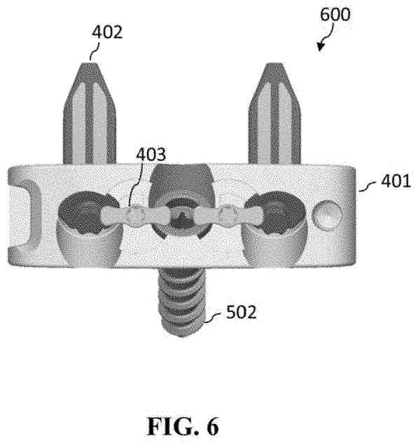

FIG. 6 depicts a view of intervertebral device 600, which is the same as intervertebral devices 400 and 500 except that it has a combination of anchors 402 and screws 502 that are inserted. FIG. 6 shows intervertebral device 600 in a closed position.

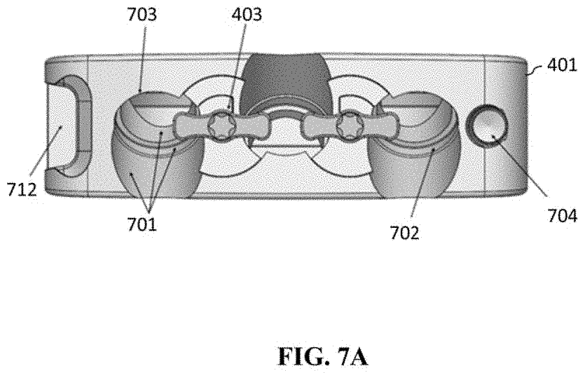

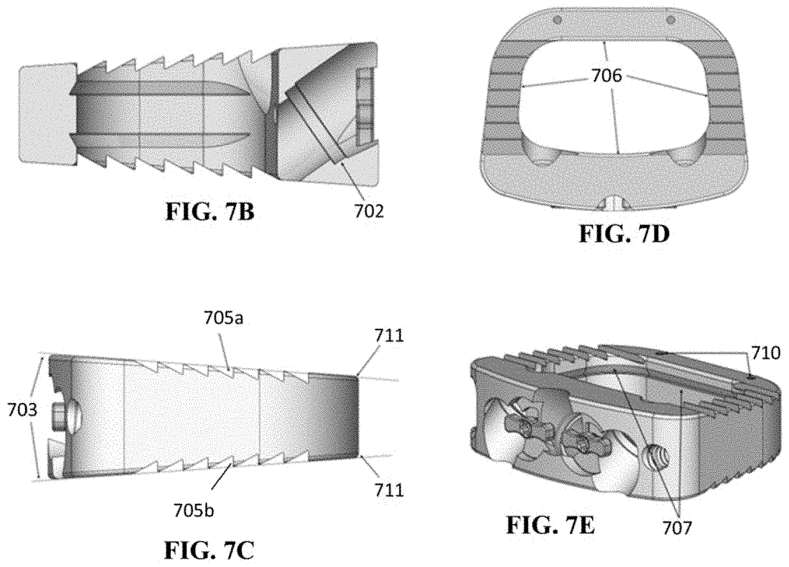

FIGS. 7A-7E depict different views of intervertebral device 400, 500, and 600 without any anchors or screws, which, in the absence of anchors and screws is the same. In this manner, the anchors 402 and screws 502 are interchangeable, i.e., the same body 401 can be used by the surgeon, who can then elect the combination of anchors 402 and screws 502 that the surgeon/practitioner wishes to use to anchor the intervertebral device in place.

Interbody cage 401 includes bores 701 (which can be cylindrical bores) that allow for the passage of screws 502 or anchors 402. The decision of which to use can be made intraoperatively. As shown in FIG. 7B, each bore 701 has an interference fit 702 with the anchor head for anchor retention within interbody cage 401. Interbody cage 401 further includes top and bottom interbody cage surfaces 703 that are generally the same constant distances away from cylindrical bores 701 despite the change in interbody cage heights. The interbody cage top and bottom surfaces 703 may be parallel or angled relative to each other.

Interbody cage 401 further includes a threaded hole 704 for a rigid attachment to an insertion device and a lateral pocket 712 on the opposite side to resist interbody cage rotation.

As shown in FIG. 7C, interbody cage 401 further includes teeth features 705a-705b on the superior and inferior surfaces to resist device expulsion. Distal tip 711 of interbody cage 401 has geometry for ease of insertion into patient.

As shown in FIG. 7D, interbody cage 401 has a window 706 that allows for autograft and/or allograft material to be added.

As shown in FIG. 7E, interbody cage 401 further includes surfaces 707 adjacent to window 706. Surfaces 707 have a geometry for increased retention of autograft and/or allograft material within the interbody cage 401. Interbody cage 401 has radiographic markers 710 to help determine implant position within the patient.

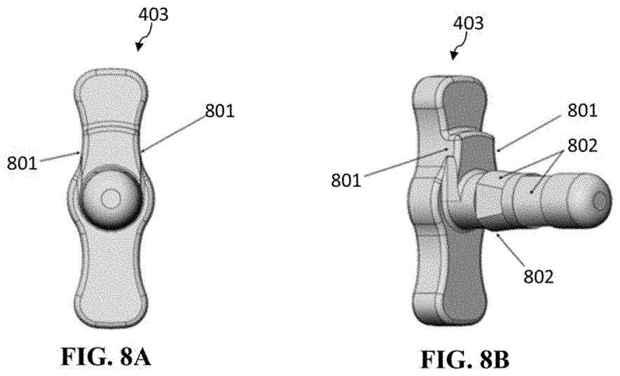

FIGS. 8A-8B depict an embodiment a locking mechanism 403 of the intervertebral device. Locking mechanism 403 includes an integrated anti-backout mechanism 802 that prevents the screws and anchor blades from migrating out of interbody cage 401. Integrated anti-backout mechanism 802 has limiting features 801 that work with the interbody cage 401 to indicate locked and unlocked positions.

FIG. 9A-9D depict an embodiment of an anchor 402 that can be used in the intervertebral device. Anchor 402 has smooth curved backside surfaces 902 for ease of insertion through cancellous and or cortical bone. Anchor 402 has porous or textured curved frontside surfaces 903 for bone in-growth into anchor 402. Anchor 402 has features 904 on the backside surfaces 902 for increased strength and instrument tracking through interbody cage 401 into cortical and cancellous bone while being implanted. Anchor 403 has a notch 905 that fits on the backside of the interbody cage's radial or arched surface 702 for proper anchor tracking and placement within the interbody cage 401. Anchor 402 has a feature 906 for anchor removal from interbody cage 401. Anchor 402 has features 907 that have an interference fit with the interbody cage's cylindrical bores 701 for anchor retention within interbody cage 401. Anchor 402 has a beveled tip 908 and a tapered tip 909 for ease of penetration.

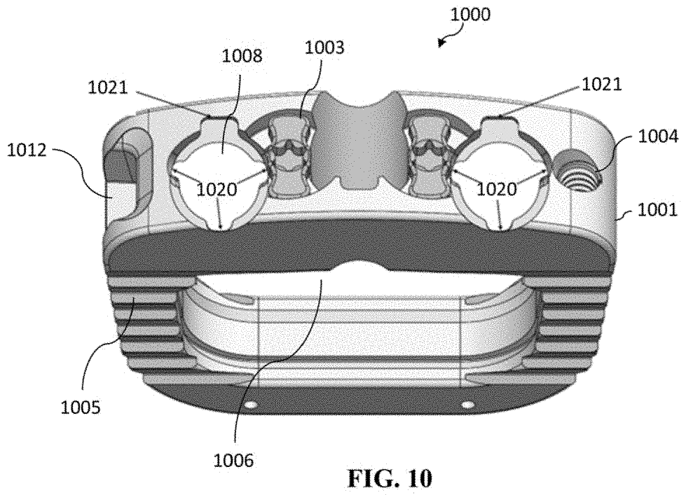

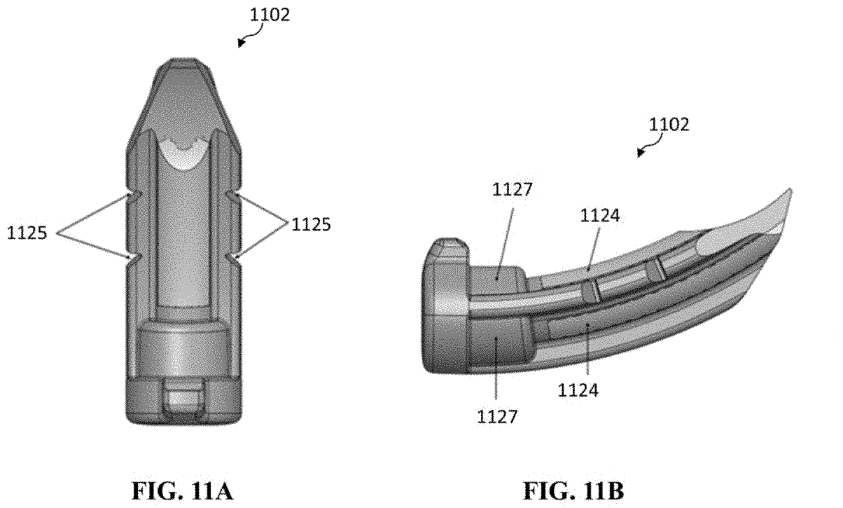

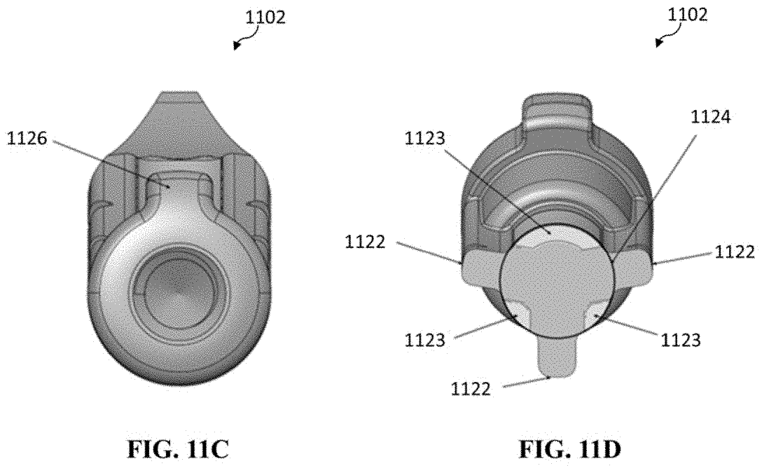

Another embodiment of the present invention is depicted in FIG. 10, which shows intervertebral device 1000 without any anchors or screws inserted. Embodiments of anchors 1102 that can be used in intervertebral device 1000 are shown in FIGS. 11A-11D and are discussed below.

Intervertebral device 1000 has a body portion 1001 (such as an interbody cage) in which the anchors 1102 are received (and which can be used by a surgeon/practitioner to anchor the intervertebral device 1000. Intervertebral device 1000 also is shown to have a locking mechanism 1003, which can be set in the open (unlocked) position or the closed (locked position). When the locking mechanism 1003 is in the open position (as shown in FIG. 10), the anchors 1102 can be inserted into the intervertebral device 1000 and anchored by the surgeon. When the locking mechanism 1003 is in the closed position, the anchors 1102 are held in place and remain anchored.

Interbody cage 1001 includes bores 1001 (which can be cylindrical bores) that allow for the passage of screws or anchors 1102. The decision of which to use can be made intraoperatively. Each bore 1001 has notches 1020 that can orient the anchor 1102 (or screw) during insertion and post-op. Moreover, each bore 1001 has a tab 1021 for visual confirmation of that the anchor 1102 (or screw) is in the proper seated position.

Intervertebral device 1000 further has similar features as discussed above for the intervertebral devices 400, 500, and 600. For instance, as shown in FIG. 10, interbody cage 1101 further includes top and bottom interbody cage surfaces that are generally the same constant distances away from cylindrical bores 1001 despite the change in interbody cage heights. The interbody cage top and bottom surfaces may be parallel or angled relative to each other.

Interbody cage 1001 further includes a threaded hole 1004 for a rigid attachment to an insertion device and a lateral pocket 1012 on the opposite side to resist interbody cage rotation. Interbody cage 1001 further includes teeth features (such as teeth features 1005 on the inferior surface) to resist device expulsion. Interbody cage 1001 has a window 1006 that allows for autograft and/or allograft material to be added. Interbody cage 1001 also has bores 1008 (which can be cylindrical bores) that allow for the passage of screws anchors 1102.

FIG. 11A-11D depict an embodiment of an anchor 1102 that can be used in the intervertebral device 1000. Anchor 1102 is similar to anchor 402 discussed and described above.

Anchor 1102 has a smooth surface 1122 that guides anchor 1102 thorough instrumentation and orients anchor 1102 when it enters interbody cage 1101 of the intervertebral device 1000. Surface 1123 allows for a porous or texture surface for bone-in growth into anchor 1102.

Anchor 1102 can have a cylindrical geometry 1124 (such as shown in FIGS. 11A-11D) for increased strength. Anchor 1102 further has notches 1125 to prevent anchor movement post-op. Anchor 1102 also has a tab 1126 and a boss 1127. Tab 1126 is for visual confirmation that the anchor 1102 is seated into the interbody cage 1101 after insertion. Boss 1127 is to prevent toggle within bores 1001.

### Anchor System Instrumentation

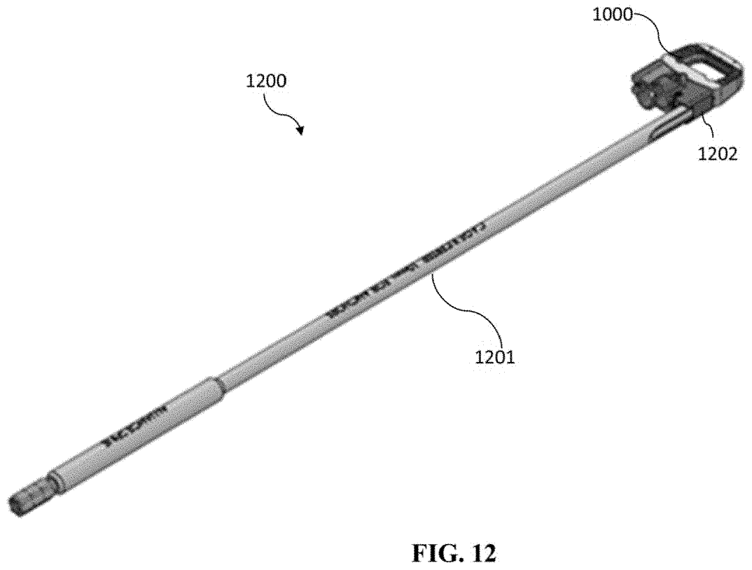

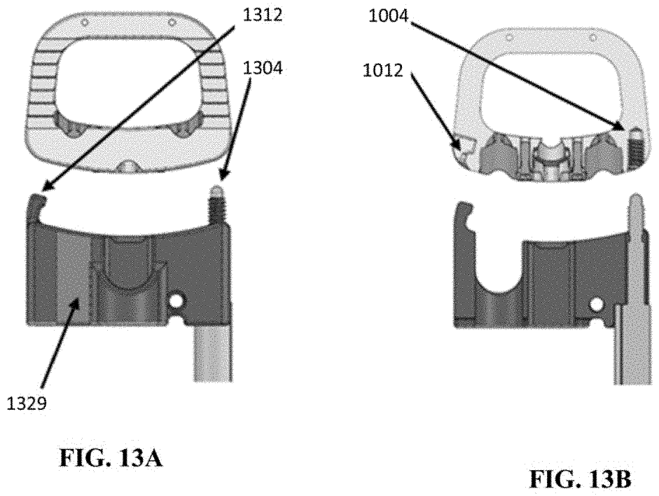

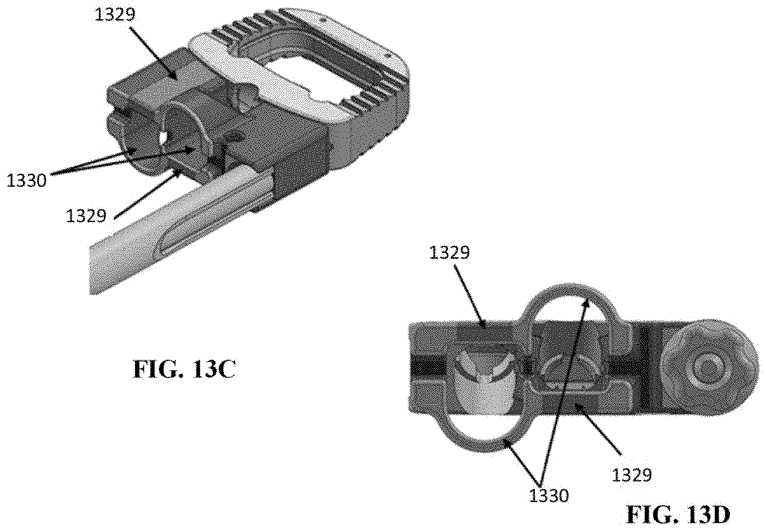

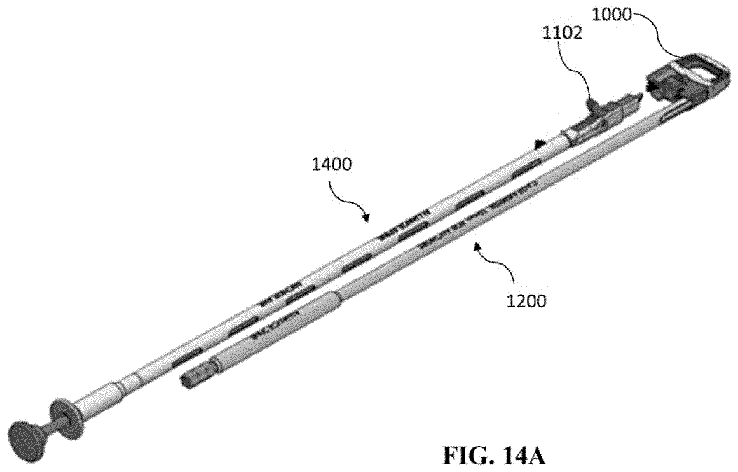

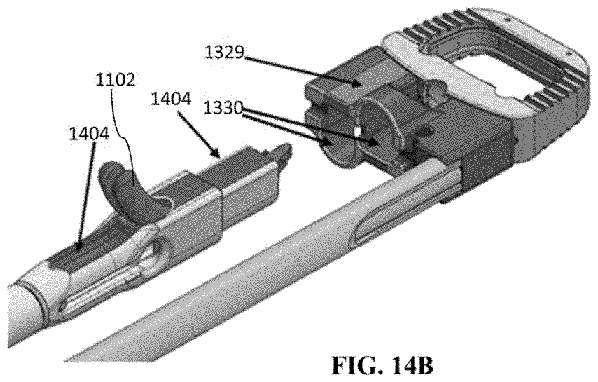

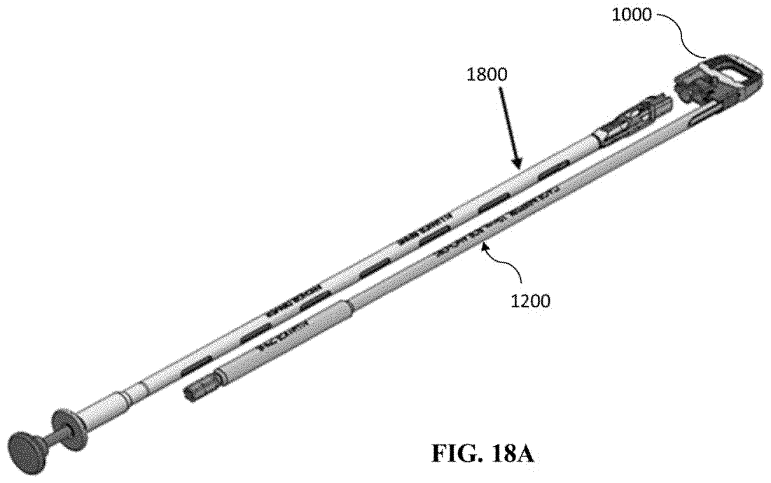

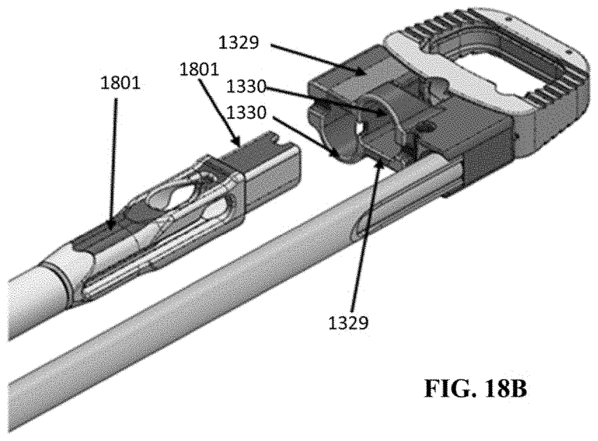

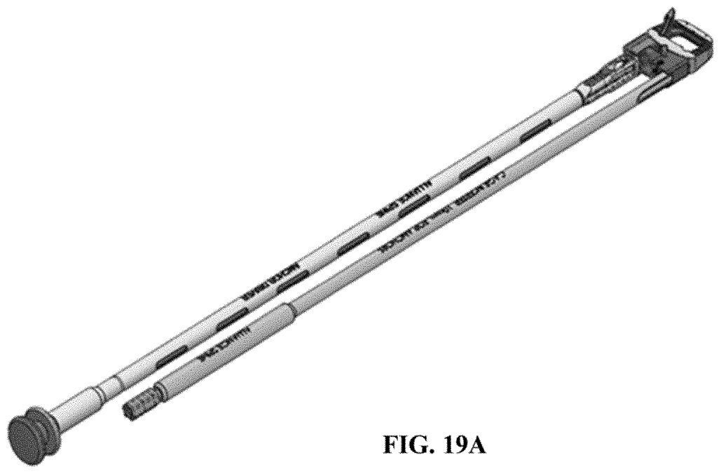

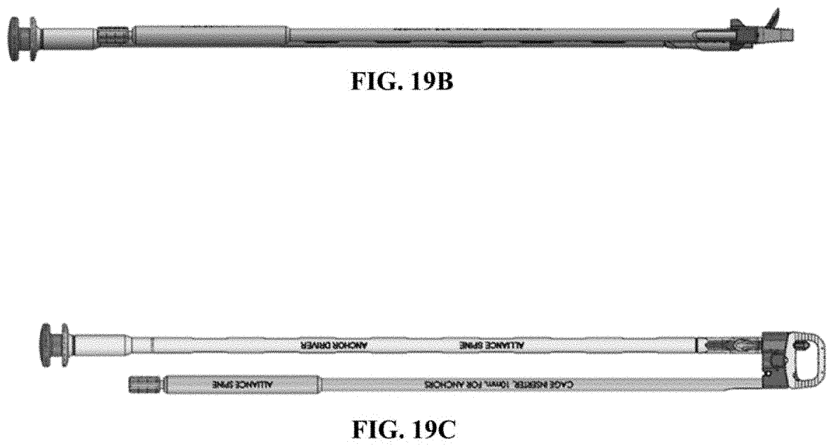

The surgeon/practitioner/clinician may utilize the intervertebral devices by insert anchors in procedures, such as in minimally invasive spine (MIL) surgical approach, i.e., perpendicular to the intervertebral device at the surgical level. Instruments associated with the intervertebral device of the present invention (such as intervertebral devices 400, 500, 600, and 1000) can be utilized. (This generally not applicable when screws are being inserted, as the screw system can utilize instruments that are not in-line with the intervertebral device). Such instruments include anchor inserters (such as shown in FIGS. 12 and 13A-13D), anchor awls (such as shown in FIGS. 14A-14B, 15A, 15C, and 16-17), and anchor drivers (such as shown in FIGS. 18A-18B and 19A-19C).

### Anchor Inserters

FIG. 12 is an anchor inserter 1200 that is coupled to intervertebral device 1000. Anchor inserter includes a shaft 1201 and head portion 1202. Shaft 1202 is an in-line anchor inserter shaft that is perpendicular to intervertebral device 1000 to enable an in-line MIS surgical approach. FIG. 13 is a magnified view of the head portion 1202 of the anchor inserter 1200 (with intervertebral device 1000 uncoupled). FIG. 13B shows a cross section of the head portion 1202 of the anchor inserter 1200 (with intervertebral device 1000 uncoupled). FIGS. 13C-13D show other magnified views of the head portion 1200 of the anchor inserter of FIG. 12 (with a coupled intervertebral device).

Head portion 1202 has attachment features 1304 and 1312 that can be used to rigidly attach (i.e., couple) anchor inserter 1200 to intervertebral device 1000 utilizing threaded hole 1004 and lateral pocket 1012. This enables shaft 1202 to be an in-line anchor inserter shaft that is perpendicular to intervertebral device 1000 to enable an in-line MIS surgical approach.

Head portion 1202 further has orientation features 1329 that orient intervertebral device 1000 allowing for it to be inserted within the patient with either one anchor pointed in a caudal direction or one anchor pointed in a cephalad direction based on surgeon preference.

Head portion 1202 further has multiple openings 1330 that are perpendicular to intervertebral device 1000 enabling in-line MIS surgical approach (perpendicular to intervertebral device/surgical level) when using the anchor awl and/or anchor driver with anchors. Multiple openings 1330 can also guide the anchor awl and/or anchor driver with anchors through the intervertebral device 1000 and into the vertebral endplates. Multiple openings 1330 can also orient the anchor awl and anchor driver.

Anchor Awls

FIG. 14A shows an anchor awl 1400 with the anchor inserter 1400 (coupled to intervertebral device 1000). FIG. 14B shows a magnified view of a portion of anchor awl 1400. Anchor awl 1400 holds (and orient) anchor 1102. Anchor awl 1400 has anchor awl orientation features 1404 that guides anchor awl 1400 into anchor inserter 1200 with its in-line shaft perpendicular to the intervertebral device 1000 in anterior direction to enable an in-line MIS surgical approach. Anchor awl orientation features 1404 further orient the anchor inserter 1400 so the surgeon knows which vertebral body the anchor 1102 is going to enter into.

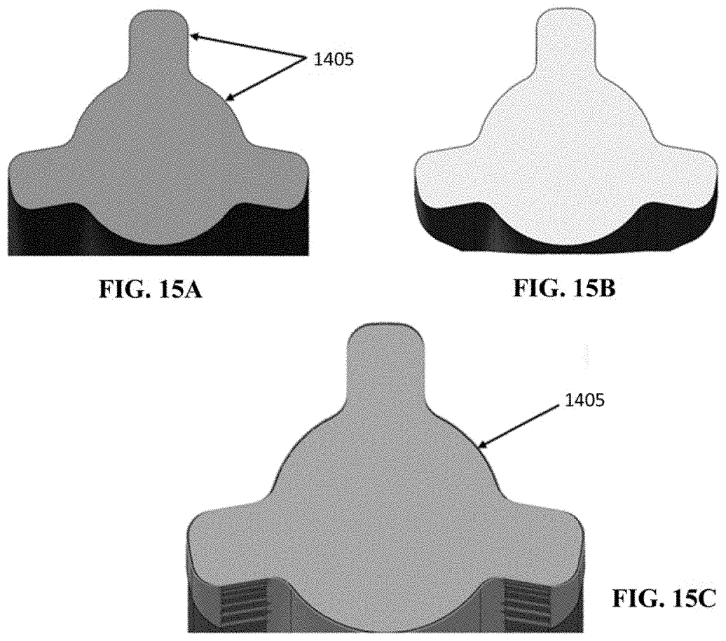

FIGS. 15A-15C show, respectively, sections through the (i) anchor awl, (ii) anchor, and (iii) anchor awl and anchor showing profiles. Anchor awl 1400 has a profile that is similar to the profile of anchor 1102 that creates an undersized opening diametrically within the vertebral body's endplate.

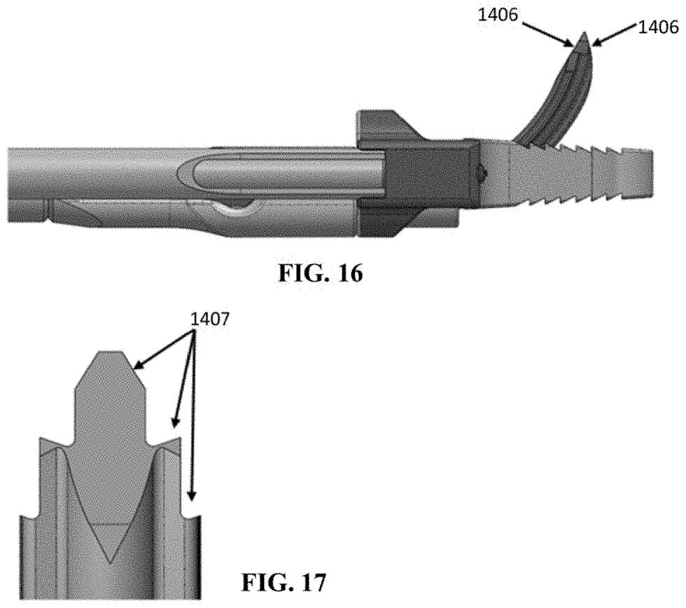

FIG. 16 shows another magnified view the portion of the anchor awl 1400. FIG. 17 depicts a magnified view of tip 1406 of anchor awl. Tip 1406 has bevels and has 1407 steps for ease of insertion into the vertebral body's endplate.

Anchor Driver

FIG. 18A shows anchor driver 1800 with the anchor inserter 1200 and intervertebral device 1000. FIG. 18B depicts a magnified view of a portion of anchor driver 1800. FIGS. 19A-19C show other views of anchor driver 1800 (fully coupled with anchor inserter 1200 and intervertebral device 1000).

Anchor driver 1800 has anchor driver orientation features 1801 that guide into anchor inserter 1200 with its in-line shaft that is perpendicular to the intervertebral device 1000 in anterior direction to enable an in-line MIS surgical approach. Anchor driver orientation features 1801 orient the anchor device 1800 with anchor inserter 1200 so the surgeon knows which vertebral body the anchor tip is going to enter into.

### Uses of Intervertebral Devices

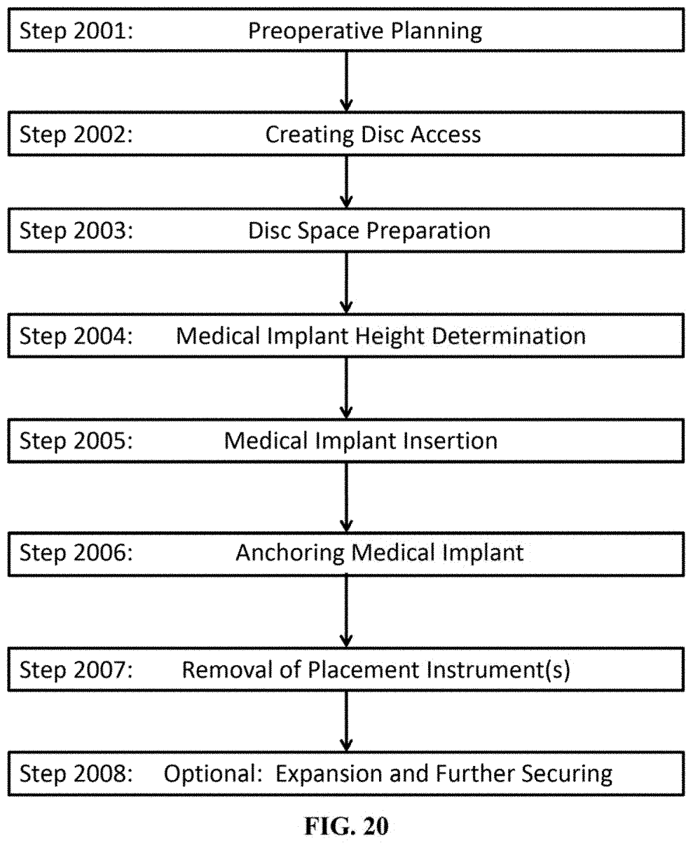

FIG. 20 is a flowchart of a surgical technique using the intervertebral device of the present invention (such as intervertebral devices 400, 500, 600, and 1000). A surgeon or other practitioner can use the intervertebral device of the present invention in a method that includes the following steps:

In step 2001, the surgeon/practitioner performs preoperative planning. This includes determination of the appropriate height of the medical implant (i.e., interbody cage) before the surgery. To achieve maximum segment height restoration, the medical implant should be selected having the largest possible height that can be safely inserted without disturbing the surrounding neural elements. Typically, the height is between 5 and 20 mm. Such preoperative planning should also take into account what the anchoring height should be. For example, if a single-opened-height controlled anchoring system is to be utilized, the surgeon/practitioner should select an medical implant that also includes the height the surgeon/practitioner desires. Further for example, if a variable-opened-height controlled anchoring system, the surgeon/practitioner should select an medical implant having a range of height that includes the height the surgeon/practitioner desires. (This preoperative planned heights of the medical implant and the anchoring system can be adjusted in step 2004 described below)

In step 2002, the surgeon/practitioner creates disc access. This, of course, depends on the surgical procedure being utilized by the surgeon/practitioner. I.e., the procedure an approach from the anterior, posterior, etc.). For the purposes of the description herein, the procedure will be oriented for an anterior approach. A person of skill in the art would understand how such procedure described below would be altered for other approaches. For instance in a typical anterior approach, the patient is placed in the prone position. From the midline anteriorly, the surgeon/practitioner would dissect the skin, subcutaneous tissues, and to expose (anteriorly) a portion of the spinal column. This exposed a disc space between adjacent vertebrae (or discs).

In step 2003, the surgeon/practitioner prepares the disc space. Using the appropriate instruments, the surgeon/practitioner removes the disc material. The surgeon/practitioner can decorticate the cartilaginous endplates from the surface of adjacent vertebral endplates until bleeding bone is obtained.

In step 2004, the surgeon/practitioner makes a height determination. Rasp trials may be used for further endplate preparation as well as to distract the vertebral space. This allows for the removal of small irregularities along the endplate better ensuring a smooth surface for medical device insertion. Rasp or smooth trials can be used to determine the appropriate size of the medical device to be inserted (and, if warranted, the surgeon/practitioner can modify which medical device to utilize so that the appropriate heights of the medical device and its anchoring system are proper). Trials also provide the surgeon/practitioner with tactile feedback as it relates to the distraction of the vertebral space. The surgeon/practitioner can select a rasp or smooth trial that corresponds to the preoperative estimated height and the prepared endplates. The surgeon/practitioner can insert the rasp or smooth trial into the disc space until the desired height is achieved. The surgeon/practitioner can confirm height and position visually and/or under fluoroscopy.

The surgeon/practitioner should then select a rasp or smooth trial that corresponds to the preoperative estimated height of the medical device, as applicable. Trials that can be selected include parallel trials and lordotic trials. The surgeon/practitioner can apply gentle impaction to ensure that the trial fits tightly and accurately between the endplates. The surgeon/practitioner can then confirm height, depth, and position under fluoroscopy. Care should be taken to protect the nerve roots, dura, and spinal cord while placing rasp trials and/or smooth trials. (This is true also when inserting the implants, including the medical device).

In step 2005, the surgeon/practitioner inserts the medical device. Optionally, and typically before such insertion of the medical device, the surgeon/practitioner can pack the grafting area of medical device with bone graft (such as autologous bone graft) in the interior space of the medical device. The surgeon/practitioner selects the medical device that corresponds to the rasp trial or smooth trial size. The medical device is inserted with the anchoring mechanism of the medical device in the closed position. The surgeon/practitioner can attach the medical device to an inserter tool and use this to insert the medical device. (A tamp can be used to control the positing of the medical device in the disc space). The surgeon/practitioner can insert the medical device into the prepared intervertebral space. Gentle impaction on the multi-tool or inserter tool will assist in correct positioning.

Once properly positioned, in step 2006, the surgeon/practitioner can then secure the medical device by placing the locking mechanism in the open position, inserting the screws and/or anchors (as selected by the surgeon/practitioner), and anchoring them to the bone. During this step the surgeon/practitioner can verify the proper placement and anchoring of the medical device. When an anchor/screw (or pair) are anchored in place, the locking mechanism through which the anchor/screw was inserted can then be moved to the closed position, so that the anchor/screw (or pair) remains in place. For anchors used in an in-line MIS surgical approach, anchor inserts, anchor awls, and anchor drivers can be utilized for positioning and securing of the anchor.

In step 2007, any instrument that was used to position the medical implant, and hold it in place while the medical implant was being anchored is removed.

In steps 2008, which are optional steps, expansion of the medical device and further securing can be performed by the surgeon/practitioner. For instance, if an expandable medical device is utilized (which is not shown in the figures, but is well known in the art), the medical device can be expanded laterally, vertically, or both, as the case may be. Moreover, the surgeon/practitioner can further secure the medical device by using fasteners (such as screws). Because the medical device is already secured and anchored, the medical device stays in place during this further securement and anchoring, which facilitates the procedure for the surgeon/practitioner.

A medical procedure kit (or set) fully supports the surgical procedure to implant the medical device using the medical implant fixation system can be supplied. Such kit can include one or more medical implant fixation devices (with medical implants, plates, and medical implant fixation instruments) and some or all of the following tools of a rasp trial, a smooth trial, another trial, inserter, and tamp.

The combination of tools and medical implant fixation systems can be pre-sterilized for ready use.

The disclosures of all patents, patent applications, and publications cited herein are hereby incorporated herein by reference in their entirety, to the extent that they provide exemplary, procedural, or other details supplementary to those set forth herein. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of approximately 1 to approximately 4.5 should be interpreted to include not only the explicitly recited limits of 1 to approximately 4.5, but also to include individual numerals such as 2, 3, 4, and sub-ranges such as 1 to 3, 2 to 4, etc. The same principle applies to ranges reciting only one numerical value, such as “less than approximately 4.5,” which should be interpreted to include all of the above-recited values and ranges. Further, such an interpretation should apply regardless of the breadth of the range or the characteristic being described.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

What is claimed is:

1. A medical implant system comprising:
(a) a plurality of fasteners comprising screws and/or anchors;
(b) a receiving member body having a plurality of openings, wherein
(i) each of the openings in the plurality of openings is capable of interchangeably receiving a screw or an anchor from the plurality of fasteners;
(c) anchor system instrumentation that is operable for rigidly attaching to the receiving member body resulting in the anchor system instrumentation being perpendicular to a midline of the receiving member body; and
(d) a plurality of locking mechanisms connected to the receiving member body, wherein
(i) each opening in the plurality of openings has at least one locking mechanism located nearby,
(ii) each of locking mechanism in the plurality of locking mechanisms is capable of being moved with an instrument between an open position and a closed position relative to the opening nearby the locking mechanism,
(iii) when the locking mechanism is in the open position, the locking mechanism does not cover any portion of the opening nearby the locking mechanism such that the fastener can be passed through the opening to secure the fastener into bone;
(v) when the locking mechanism is in the closed position, a portion of the locking mechanism covers a portion of the opening such that the fastener cannot pass through the opening, such that the fastener is prevented from becoming unsecured from the bone.

2. The medical implant system of claim 1, wherein the anchor system instrumentation comprises one or more instruments selected from a group consisting of anchor inserters, anchor awls, anchor drivers, and combinations thereof.

3. The medical implant system of claim 1, wherein the anchor system instrumentation comprises an anchor inserter, an anchor awl, and an anchor driver.

4. The medical implant system of claim 1, wherein the receiving member body is an interbody cage.

5. The medical implant system of claim 1, wherein the anchors have anchor blades.

6. The medical implant system of claim 1, wherein the openings are cylindrical.

7. The medical implant system of claim 1, wherein the openings have notches that orient the anchors during insertion into the receiving member body.

8. The medical implant system of claim 1, wherein the openings and the anchors each have a tab for visual conformation that the anchor has been seated in position within the opening.

9. The medical implant of claim 1, wherein the anchor has anchor notches to prevent anchor movement after securing the anchor to the bone.

10. The medical implant of claim 1, wherein the anchor has a surface that allows for a porous or textured surface for bone-in growth into the anchor.

11. The medical implant of claim 1, wherein the receiving member body has a window so that a bone growth inducing substance can be utilized with the medical implant or so that fusion can be more readily viewed.

12. The medical implant system of claim 1, wherein each of the locking mechanisms has an integrated anti-backout mechanism.

13. The medical implant system of claim 12, wherein the anti-backout mechanism prevents the screw or blade of the anchor from migrating out of the interbody cage.

14. The medical implant system of claim 12, wherein the anti-backout mechanism is operable for interacting with the receiving member body to indicate the open position and the closed position.

15. A method comprising the steps of:
(a) selecting a medical implant system having a plurality of fasteners, a receiving member body having a plurality of openings, and a plurality of locking mechanisms, wherein the plurality of fasteners comprise screws and anchors;
(b) selecting a first fastener from the plurality of fasteners, wherein the first fastener is a screw or an anchor;
(c) while a first locking mechanism is in an open position relative to a nearby first opening in the plurality of openings, inserting the first fastener through the nearby first opening such that the first fastener is received into the receiving member body;
(d) securing the first fastener into bone;
(e) repeating steps (b)-(d) for additional fasteners in the plurality of fasteners to secure the receiving member body, wherein for each additional fastener, the additional fastener selected can independently be a screw or an anchor from the plurality of fasteners; and
(f) moving the locking mechanisms from the open positions to the closed positions, wherein a portion of the first locking mechanism covers a portion of the first opening such that the fasteners cannot pass through the openings, and such that the fasteners are prevented from becoming unsecured from the bone.

16. The method of claim 15 further comprising rigidly attaching anchor system instrumentation to the receiving member body resulting in the anchor system instrumentation being perpendicular to a midline of the receiving member body.

17. The method of claim 16, wherein the anchor system instrumentation comprises one or more instruments selected from a group consisting of anchor inserters, anchor awls, anchor drivers, and combinations thereof.

18. The method of claim 16, wherein the anchor system instrumentation comprises an anchor inserter, an anchor awl, and an anchor driver.

19. The method of claim 15, wherein the receiving member body is an interbody cage.

20. The method of claim 15, wherein the anchors have anchor blades.

21. The method of claim 15, wherein the openings are cylindrical.

22. The method of claim 15 further comprising orienting the anchors during insertion into the receiving body utilizing one or more notches in the opening.

23. The method of claim 15 further comprising visually confirming that the anchor has been seated in position within the opening utilizing tabs in the opening and the anchor.

24. The method of claim 15, wherein the anchor has anchor notches to prevent anchor movement after securing the anchor to the bone.

25. The method of claim 15, wherein the anchor has a surface that allows for a porous or textured surface for bone-in growth into the anchor.

26. The method of claim 15, wherein
(a) the receiving member body has a window, and
(b) the method further comprises utilizing the window to (i) provide a bone growth inducing substance, (ii) view fusion, or (iii) both.

27. The method of claim 15, wherein each of the locking mechanisms has an integrated anti-backout mechanism.

28. The method of claim 28, wherein the anti-backout mechanism prevents the screw or blade of the anchor from migrating out of the interbody cage.

29. The method of claim 15, wherein the anti-backout mechanism interacts with the receiving member body to indicate the open position and the closed position.
